(19) United States
(12) Patent Application Publication
(10) Pub. No.: US 2004/0147851 A1
(43) Pub. Date: Jul. 29, 2004

(54) Device and method for monitoring respiratory movements

(76) Inventor: Simon Bignall, Middlesex (GB)

Correspondence Address: Flynn Thiel Boutell & Tanis, 2026 Rambling Road, Kalamazoo, MI, 49008-1631, US

(21) Appl. No.: 10/485,135

(22) PCT Filed: Jul. 29, 2002
(86) PCT No.: PCT/GB02/03478

(30) Foreign Application Priority Data
Jul. 30, 2001 (GB) 0118524.8

(51) Int. Cl. A61B 5/08

(52) U.S. Cl. 600/534

(57) ABSTRACT

A device sensor (9) comprising a piezoelectric bar or rod monitors respiratory movements by picking up changes in the abdomen's curvature in the cephalocaudal direction.

The present invention is concerned with a device for monitoring respiratory movements.

In medical environments or situations it may be necessary to monitor a patient's respiratory movements. Respiratory movement information may be helpful in medical diagnosis as well as physiological evaluation and monitoring. For example, newborn infants and babies in the first year of life often require respiratory monitoring or support when they are ill.

One existing method (the Wright respiration monitor) uses a foam-filled capsule which is taped to the abdomen and responds to changes in curvature. The monitor is taped using an adhesive tape which is stretched over the capsule and stuck to the skin on either side of the capsule. The monitor registers an increase in pressure when the surface becomes more curved and the capsule becomes compressed between the skin and tape.

Other existing methods use bands containing or affixed to transducers which encircle the abdomen and which respond to diaphragmatic descent by recording changes in tension in an encircling band, or in inductive changes in a loop of wire, or the position of a Hall effect component next to the skin.

Current methods for the monitoring of infant respiration tend to be unreliable in long-term use because:

a) they fall off or move on the patient's body (when worn under clothing, for example) and do not let the carer know this;

b) they may be placed inaccurately, giving both false positive and false negative readings;

c) they are difficult to site in order to give a good indication of respiration;

d) they do not give feedback to the carer as he/she tries to ensure that the transducing sensor is properly applied; and/or

e) they are prone to respond to non-respiratory influences on the shape of the abdomen or chest, such as writhing, squirming or any movement which uses or affects the shape of the abdominal wall musculature.

Furthermore current monitoring methods do not have a sufficiently high frequency response to allow their use during HFOV (high frequency oscillatory ventilation) in which mode of mechanical ventilation air is pumped in and out of the lungs at 5-15 Hz.

The present invention, in its various aspects, provides devices, apparatus and/or methods as defined in the independent claims to which reference is now made. Preferred features of the invention are set out in the dependent claims.

The invention in a first aspect provides a device for monitoring respiratory movements in a subject, comprising a transducer responsive to changes in a first direction but substantially unresponsive to changes in curvature in a second direction substantially orthogonal to the said first direction.

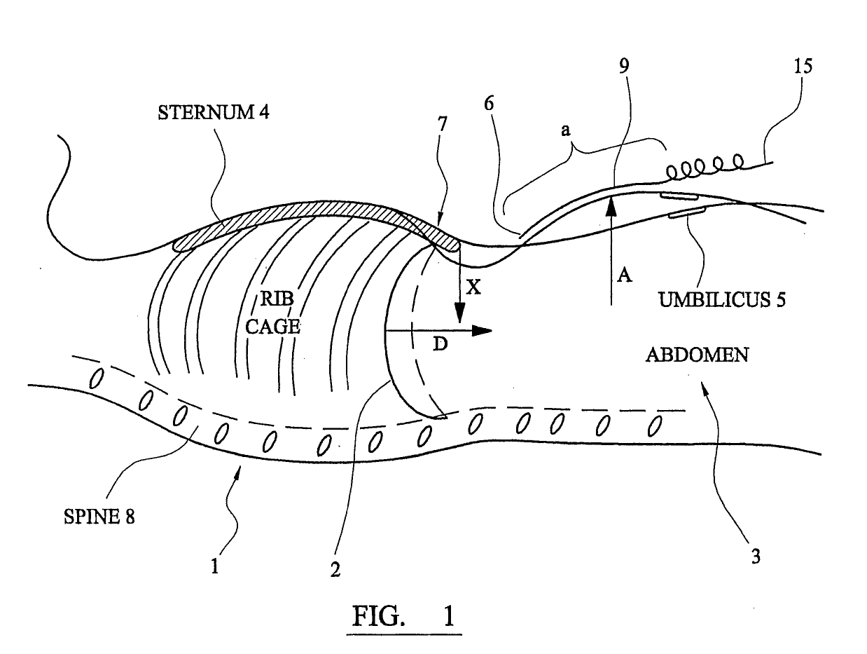

Subjects such as human infants rely on their diaphragm 2 (see FIG. 1) to inspire, placing much less reliance on the, chest wall musculature and accessory muscles of respiration than do adults and older children. This means that an accurate indication of inspiration may be gained from an examination of the movement of the diaphragm 2. When the diaphragm starts to descend at the very start of inspiration, it flattens from its resting, curved shape and pushes against the abdominal contents 3. This downward force causes the upper part of the abdomen 3 to bend in the cephalocaudal (head-to-toe) direction D, shortening the effective radius of curvature of the upper abdomen between the lower end of the sternum 4 (known as the xiphisternum 7 which is another, smaller piece of bone attached to the lower end of the sternum, or breastbone) and the umbilicus 5 (see FIG. 1).

The present invention in its first aspect allows one to pick up changes in the abdomen's curvature in the cephalocaudal direction caused by diaphragmatic movements but will not pick up or will differentiate these from abdominal movements caused by non-respiratory movements and general muscular activity. The present invention therefore makes for accurate monitoring of diaphragmatic movements.

In a preferred embodiment of the invention in its first aspect, the transducer is supported on an adhesive strip for fixing to the subject.

The support of the transducer on an adhesive strip allows for easy and secure locating and fixing of the device.

In a preferred embodiment of the invention in a first aspect, the adhesive strip is a woven fabric support element with an adhesive patch.

The woven element helps to ensure flexibility sufficient to maintain contact with the underlying skin. The transducer will itself be arranged within short transverse fibres of the woven element so that the majority of the forces acting upon the transducer will be influences by cephalocaudal deformation of the upper abdominal skin, caused by the onset of diaphragmatic excursion.

In a preferred embodiment of the invention the adhesive strip includes at least two adhesive patches of differing adhesiveness such that, when active, a first adhesive patch provides a temporary fixing allowing the device to be moved between fixing positions if so desired and the second adhesive patch provides a stronger fixing which does not allow the device to be moved between fixing positions.

The provision of adhesive patches of differing adhesiveness allows one to use an adhesive patch of relatively low adhesiveness as a temporary fixation for initial positioning and re-positioning of the device until it is confirmed that it is correctly sited. A second adhesive patch of higher adhesiveness can then be used for semi-permanent fixation of the device once it is correctly sited. The arrangement allows one to meet the apparently conflicting demands of a fixing which can be easily adjusted until the device is correctly positioned and a fixing whereby the device cannot easily be dislodged once it is correctly positioned and the patient is being monitored.

In a preferred embodiment, the device further comprises a display coupled to the transducer and mounted on or in the immediate proximity of the transducer.

The provision of a display on or in the immediate proximity of the transducer helps to ensure that whoever is placing or monitoring the device is easily able to monitor the transducer output and hence the need for adjustment of the position of the sensor or the sensitivity of the associated circuitry in order to achieve recognition of the diaphragmatic signal as early as possible during descent of the diaphragm. When the indicator and the sensor are in the same site, the user can readily compare the physical movement of the upper abdomen with the display from the sensor circuitry to ensure by observation that the circuitry produces its signal corresponding to the onset of respiration in the correct phase of respiration and at the start of inspiration. This is particularly important for long-term, unattended recording of respiration during which transducers may be moved or become detached and no longer be placed over the point of maximum curvature change with diaphragmatic descent.

The location of the display on or near the transducer and hence, when in use, on or near the relevant part of the patient also helps to ensure that changes in status are easily recognised and related to the right patient and part of the patient's anatomy. A change in a display on the patient in the relevant anatomical position is more likely to be noticed and acted upon than a change on a remote display.

The invention in a second aspect provides a device for monitoring respiratory movements comprising a sensor for monitoring a subject's abdominal and/or chest wall movements and fixing means for fixing the device to the subject's abdomen and/or chest wall wherein the device includes location markings for alignment relative to specified features of the subject's abdomen so as to position or locate the device on an abdomen and/or chest wall before fixing it thereto.

The provision of a location template allows for easy and accurate location of the device.

The invention in a third aspect provides a method for monitoring respiratory movements in a subject comprising monitoring movement of the subject's abdomen in the cephalocaudal direction.

As discussed above in connection with the invention in its first aspect, this allows one to accurately determine the onset of breathing. The invention is particularly useful for determining the onset of an infant's inspiration which is heavily dependent on movement of their diaphragm because it can sensitively monitor diaphragm movements while note being affected by non-inspiratory movements and general muscle activity.

By way of example, preferred embodiments of the invention will now be described with reference to the accompanying figures in which:

FIG. 1 is a side view of an infant's thorax and abdomen illustrating changes in curvature in the upper abdomen during respiration;

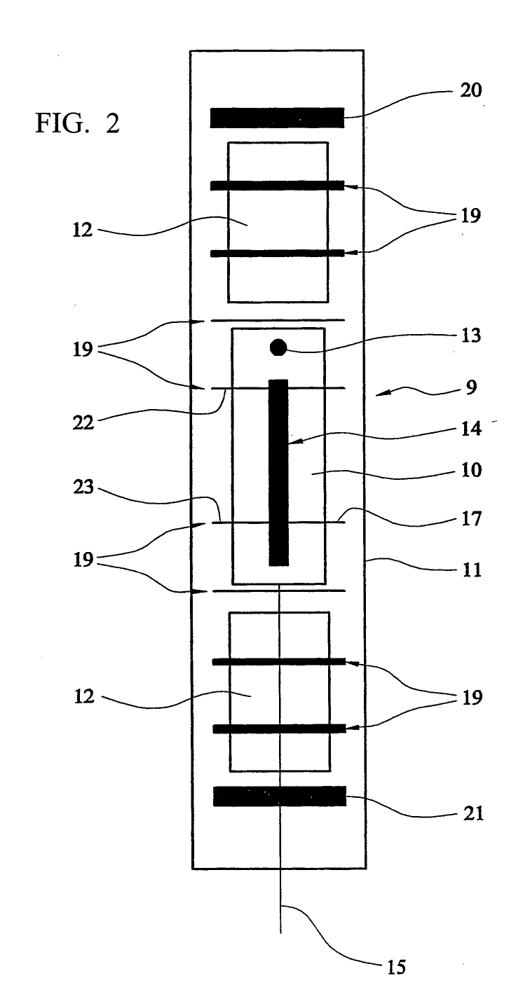

FIG. 2 is a schematic illustration of aspects of a sensor embodying the invention;

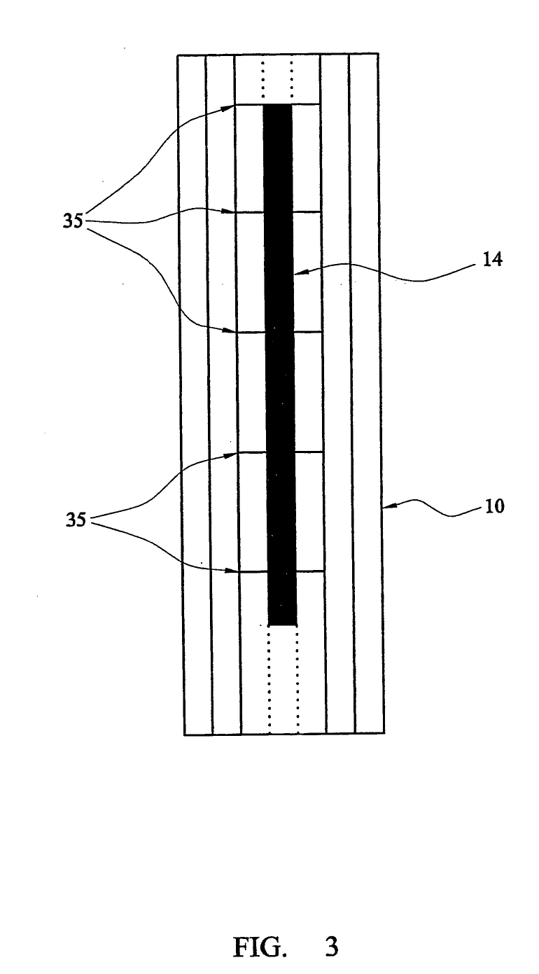

FIG. 3 is a schematic illustration of other aspects of a sensor embodying the invention;

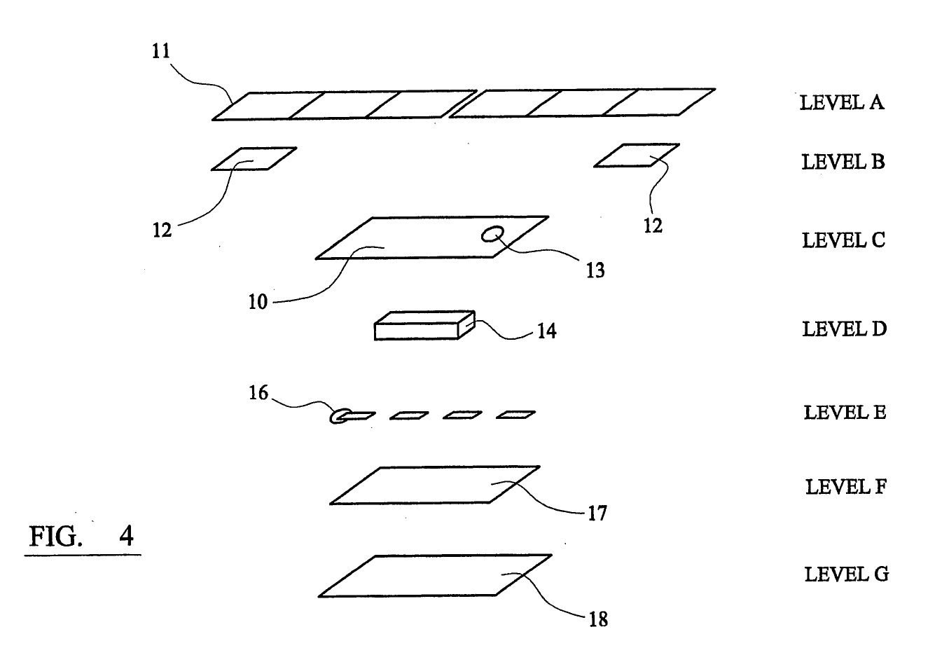

FIG. 4 is an exploded view of the elements of a sensor embodying the invention;

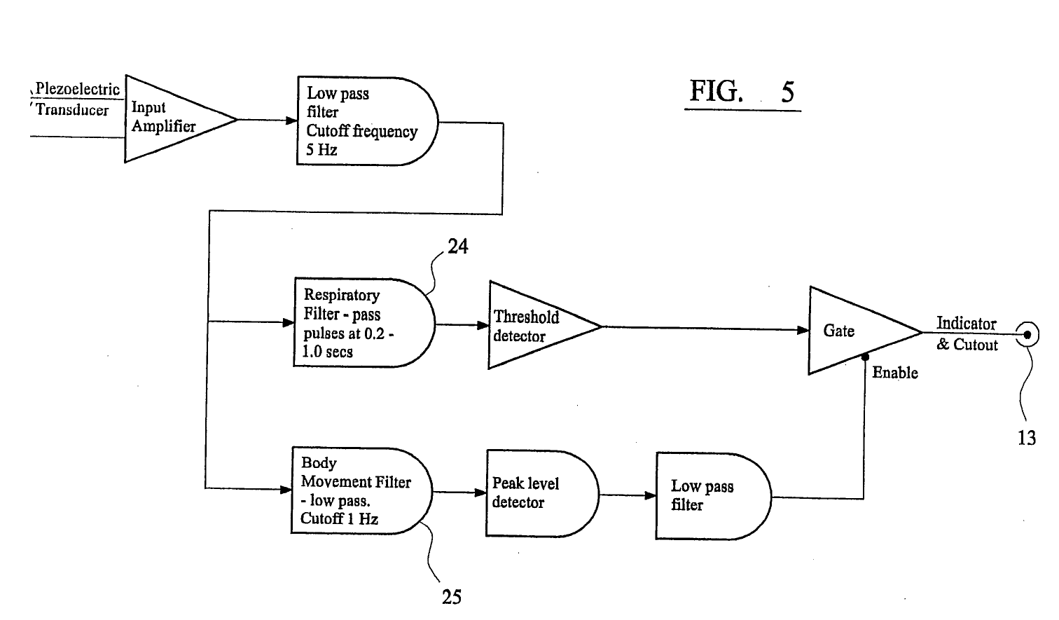

FIG. 5 is a schematic view of circuitry for use with the sensor of FIGS. 2 to 4;

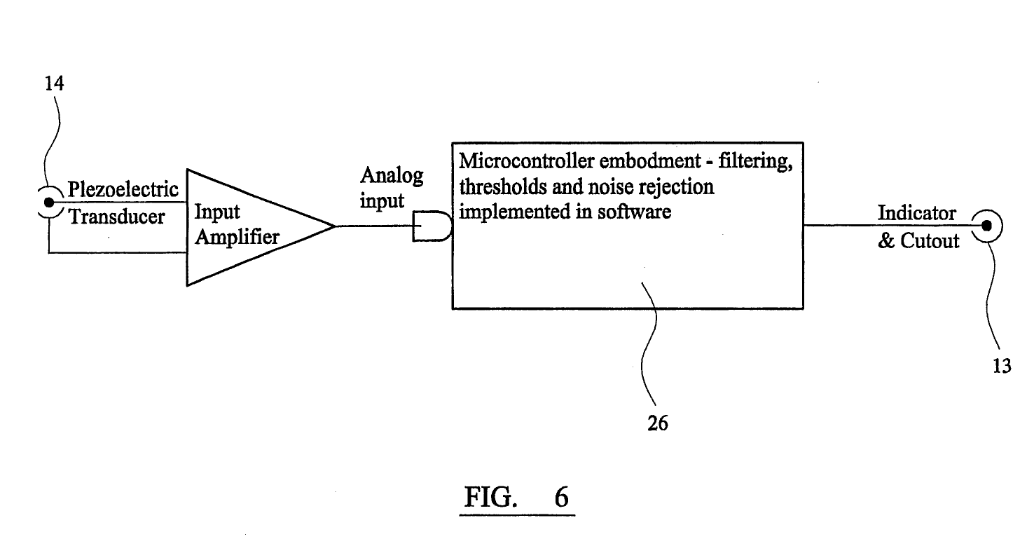

FIG. 6 is a schematic view of alternative circuitry for use with the sensor of FIGS. 2 to 4; and

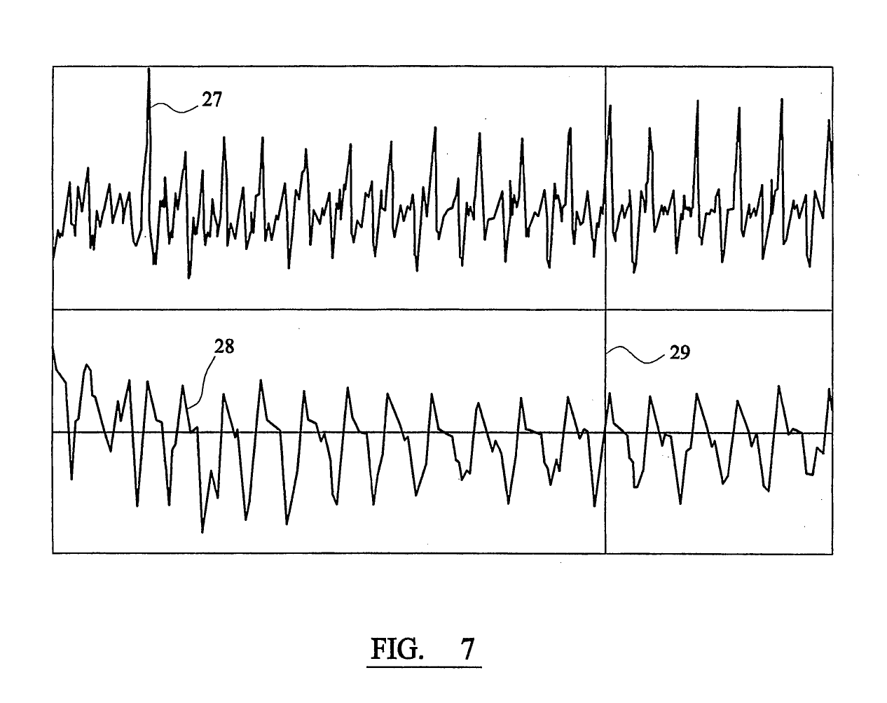

FIGS. 7 to 9 are sample outputs from apparatus including the present invention.

Embodiments of the present invention are particularly suitable for monitoring respiratory movements in infants or babies. Preferred embodiments will therefore be described in connection with human infants although the device may also be used to monitor respiratory movements in adult humans and animals.

Human infants 1 rely on their diaphragm 2 to inspire, placing much less reliance on the chest wall musculature and accessory muscles of respiration than do adults and older children. This means that an accurate indication of inspiration is readily gained from an examination of the movement of the diaphragm 2. When the diaphragm starts to descend at the very start of inspiration, it flattens from its resting, curved shape and pushes against the abdominal contents 3. This downward force causes the upper part of the abdomen 3 to bend in the cephalocaudal (head-to-toe) direction D, shortening the effective radius of curvature of the upper abdomen between the lower end of the sternum 4 (known as the xiphisternum 7 which is another, smaller piece of bone attached to the lower end of the sternum, or breastbone) and the umbilicus 5 (see FIG. 1).

Thus, the moment of onset of diaphragmatic descent, a very early point in spontaneous inspiratory effort, can be detected by any method which will respond to an increased curvature in the upper abdomen.

FIG. 1 is a side view of infant thorax and abdomen to show changes in curvature in the upper abdomen 2 occasioned by the initial descent of the diaphragm 2 moving in direction of arrow marked “D”). The position of the diaphragm 2 after the onset of inspiration by the infant is shown as a dashed line and the corresponding position of the upper abdominal surface 6 is shown as a solid line. The xiphisternum 7 at the lower end of the sternum is drawn towards the spine 8 (directional arrow marked “X”) as the diaphragm muscle fibres shorten and the upper abdominal skin 6 is forced outwards, thus increasing the curvature of the interrogated-surface (area interrogated by new sensor area marked “a” and directional arrow marked “A”).

The increase in curvature of the upper abdomen illustrated in FIG. 1 may be affected by non-inspiratory and general muscular activity. The oblique abdominal muscles are situated laterally in the abdominal wall and are often involved in active expiration. Contraction of these muscles will tend to change the curvature in non-cephalocaudal directions. In order to pick up inspiratory activity specifically, therefore, it is necessary to ensure that any transducer is carefully sited and tends to respond preferentially to the specific changes in abdominal wall curvature caused by the action of the diaphragm.

FIG. 2 illustrates a sensor 9 comprising a piezoelectric bar or rod. This piezoelectric element (see FIGS. 3 and 4) is supported on a fabric strip 10.

The fabric strip 10 includes transverse fibres which stiffen the strip in the lateral plane but maintain deformability in the cephalocaudal or another specified plane.

The sensor construction (see FIG. 4) is a layered construction. The device includes the following layers:

a) transparent template layer 11: this might be made of a transparent polymer material (e.g. polypropylene) which is biocompatible;

b) temporary fixing pads layer 12: the adhesive might be a hydrogel with low adhesiveness;

c) fabric layer 10 with integral display LED 13: LED (made of e.g. woven polyester);

d) piezoelectric element 14 and cables or wires with insulation;

e) flexible enclosing insulation layer 16 (e.g. silicone rubber) for piezoelectric element 14);

f) hydro-gel strip 17 with strong adhesiveness for semi-permanent fixation to skin; and

g) backing film 18 (e.g. polyethelene) for adhesive strip.

The upper surface is defined by a transparent biocompatible material such as polypropylene on which location markings 19 have been made.

The markings 19 on the template (see FIG. 2) represent the relative position of the lower end of the sternum (found by palpation) and the umbilicus (umbo) which are separated by a distance which varies with body length, itself closely corresponding to birth weight in the newborn baby. At a weight of 5 Kg (the largest infant likely to be found in a newborn nursery) the distance between the solid bar on the template marked “STERNUM” 20 and that marked “UMBO” 21 is approximately 10 cm. Thus for a baby of this weight, the template 11 and its associated sensor 9 with the underlying piezoelectric sensing element 14 would be applied with the two bars 20, 21 at either end overlying the lower end of the sternum 4 and the upper edge of the umbilicus 5 in order to achieve fixation halfway between. In a baby of only 500 g birthweight the distance between the umbo and sternum is typically only 2 cm, corresponding to the innermost, lightest bars 22, 23 on the template 11. In a baby of 5000 g birthweight this distance was 10 cm. Thus, for any baby between 500 g and 5000 g weight, it is possible to apply the template by choosing those corresponding bars 19 (at 1 cm intervals) from either end which cover the areas of skin closest to the lower end of the sternum 4 and the upper border of the umbilicus 5, respectively. These measurements were taken from 10 newborn babies undergoing intensive care. For older babies, a longer template might be constructed based on similar measurements.

The output from the piezoelectric element 14 varies as a patient breathes and the diaphragm 2 moves. FIGS. 5 and 6 illustrate arrangements for coupling the piezoelectric transducer 14 to an on-device indicator such as the display LED 13 and an output which may be coupled to a screen which tracks changes in output over time and thereby tracks changes in breathing movements over time.

In FIG. 5 (a completely analogue embodiment of the circuitry required) the piezoelectric transducer output is filtered using a filter 24 designed to pass waveforms at the likely respiratory rate (i.e. resembling a sinusoidal pulse with rise time of 0.1-0.5 seconds)— all frequencies above 5 Hz and below 1 Hz can be rejected—and a second parallel filter 25 designed to trigger on gross bodily movement with a high frequency cut-off at around 1 Hz. This latter filter 25 (which ideally uses an RMS or peak level detector [diode and capacitor] on its output) is used to gate the respiratory pulses so that in the event of gross movement detection (which renders the identification of spontaneous inspiration unreliable) the gate is inhibited and the detection signal is not made available.

FIG. 6 shows a schematic of mixed analog/digital circuitry which uses software embedded in a microcontroller 26 to perform these functions and which would allow the development of a user interface for varying those parameters relating to the detection of spontaneous inspiration in response to e.g. varying body weight.

FIG. 7 is a sample output display in which the upper trace 27 represents the output from a prototype piezoelectric transducer on the upper abdomen of a 29 week gestation infant aged 81 days during an ethically approved clinical research protocol. The lower trace 28 represents the output from an abdominal pressure capsule taped next to the piezoelectric transducer on the skin. Both signals have been lowpass filtered (6 Hz) to show equivalent characteristics. In both traces, the onset of diaphragmatic descent 29 is shown as a rapid upward displacement (i.e. that marked on both traces by vertical line 29). It can be seen that the respiratory signal is complex in this infant with serious lung disease and that non-respiratory and active expiratory components are present. Despite this, the new transducer was able to show clearly the onset of true inspiratory effort (sharp upstroke e.g. at vertical line marked on the traces). The abdominal capsule, however, with a lower frequency response and its tendency to respond to changes in curvature not induced by diaphragmatic descent, is more difficult to interpret.

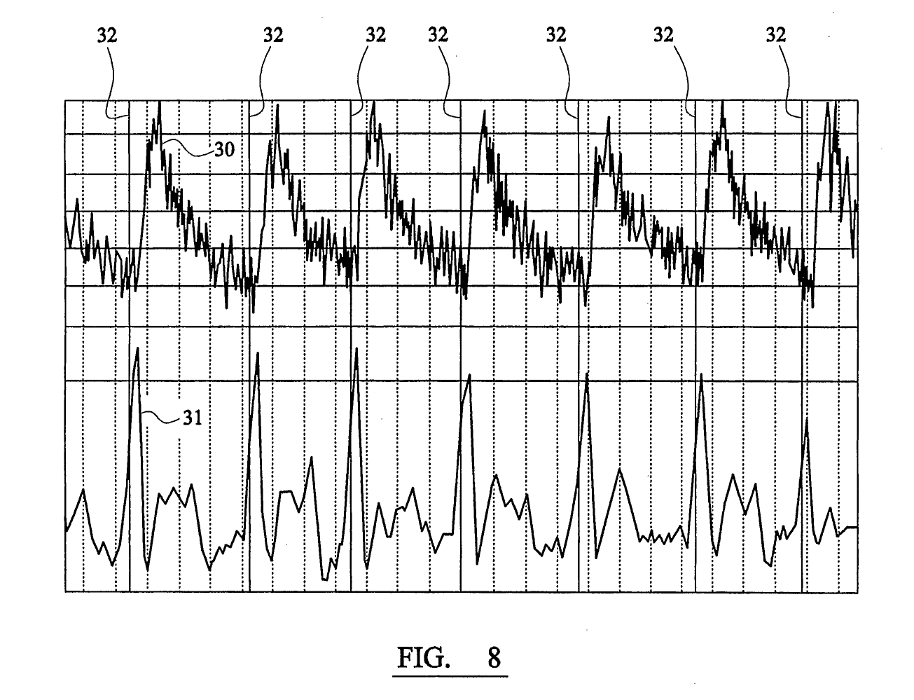

FIG. 8 shows the new transducer being used to trigger the action of a non-invasive ventilator in the same infant. The upper trace 30 (airway pressure in cm. Water, blue line) is taken from the non-invasive ventilator device, which sits in the infant's nose. The device works partly by turbulent fluidics and this accounts for the apparently noisy signal. As the device is triggered, the airway pressure rises from around 6 to 8 cm water in order to assist in lung inflation. The corresponding output from the new transducer is shown in the lower trace 31 (uncalibrated voltage) and the fiducial points at which the computer algorithm detected the onset of spontaneous inspiration are shown as vertical bars 32 marked “AC”. The device was then triggered to provide extra gas into the nose within 100 ms of diaphragmatic descent.

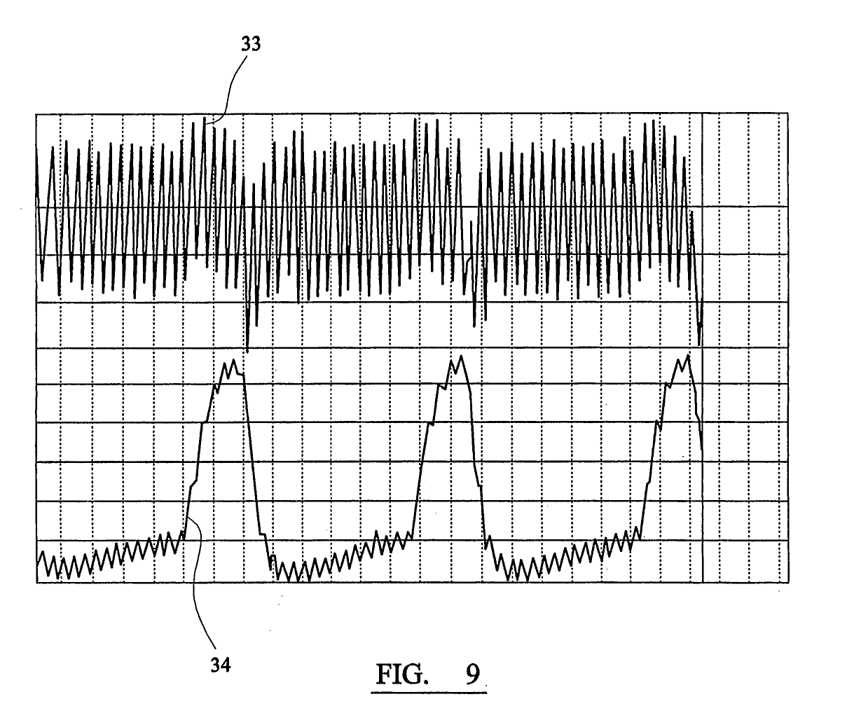

FIG. 9 shows the new transducer operating in the laboratory when a commercial oscillating ventilator was attached to a 2-litre, air-filled bag in order to bring about a roughly 0.1% change in volume at a rate of 15 Hz (in order to emulate high-frequency oscillatory ventilation). Pulses of pressure were also delivered to the bag system 40 times per minute, thus causing low pressure plateaux (marked PEEP [positive end-expiratory pressure]) and higher pressure inflations (marked INF [inflation]). This represents the most complex form of respiratory support given to the newborn. The upper trace 33 represents the output from the piezoelectric transducer. The lower trace 34 represents the output from an abdominal pressure capsule taped next to the piezoelectric transducer on the surface of the 2-litre bag. Both signals were low-pass filtered to 50 Hz. Note the ability of the new transducer to track the high-frequency oscillations faithfully during either phase of the pressure plateaux.

The higher frequency response of the new transducer will allow vibration of the upper abdomen or chest wall to be determined during HFOV. When air no longer enters the lining with each cycle of mechanical vibration during HFOV (i.e. the airway is partly or completely blocked) vibrations will reduce and an alarm may be activated to alert the carer.

1. A device for monitoring respiratory movements in a subject comprising a transducer responsive to changes in curvature in a first direction, but substantially unresponsive to changes in curvature in a second direction substantially orthogonal to the first direction.

2. A device according to claim 1 wherein the transducer, when placed on a subject's abdomen and/or chest wall, may respond to changes in curvature of the subject's abdomen and/or chest wall.

3. A device according to claim 2 wherein the transducer may respond to changes in curvature in the cephalocaudal direction.

4. A device according to any preceding claim wherein the transducer is supported on an adhesive strip for fixing to the subject.

5. A device according to claim 4 wherein the adhesive strip comprises a woven fabric support element with an adhesive patch.

6. A device according to any preceding claim wherein the transducer is responsive to changes in applied pressure.

7. A device according to any preceding claim wherein the transducer is a rod-shaped piezoelectric element.

8. A device according to claim 5 and claim 7 wherein the rod-shaped piezoelectric element has its longitudinal axis substantially parallel to fibres of the woven fabric.

9. A device according to claim 8 including fibres or elements in the woven fabric transverse to the longitudinal axis of the piezoelectric element such that the fabric is easily deformable in planes essentially orthogonal to the surface of the fabric and parallel to the piezoelectric element's longitudinal axis, and less easily deformable in a plane essentially orthogonal to the surface of the fabric and orthogonal to the piezoelectric element's longitudinal axis.

10. A device according to any preceding claim wherein the adhesive strip includes at least two adhesive patches of differing adhesiveness such that, when active, a first adhesive patch provides a temporary fixing allowing the device to be moved between fixing positions if so desired and the second adhesive patch provides a stronger fixing which does not allow the device to be moved between fixing positions.

11. A device according to any preceding claim further comprising a display coupled to the transducer and mounted within, on or in the immediate proximity of the transducer.

12. A device according to claim 11 wherein the display is one or more LEDs and/or a screen.

13. A device for monitoring respiratory movements comprising a sensor for monitoring a subject's abdominal and/or chest wall movements and fixing means for fixing the device to the subject's abdomen and/or chest wall wherein the device includes location markings for alignment relative to specified features of the subject's abdomen and/or so as to position or locate the device on an abdomen and/or chest wall before fixing it thereto.

14. Device according to claim 13 wherein the location markings are for alignment relative to anatomical features of a subject's abdomen.

15. Device according to claim 14 wherein the location markings are for alignment relative to the sternum and/or umbilicus.

16. Device according to any of claims 13 to 16 including a number of sets of location markings, each set corresponding to a subject of a pre-determined size or weight.

17. Device according to any of claims 13 to 16 and any of claims 1 to 18.

18. Apparatus including a movement monitoring device according to any of the preceding claims, said apparatus being for monitoring effects of high frequency oscillatory ventilation when ventilation is via an open, partially closed, or fully closed airway by monitoring changes in the amplitude of vibrations transmitted to the chest or abdominal wall as patency of the airway varies.

19. A method for monitoring respiratory movements in a subject comprising monitoring movement of the subject's abdomen in the cephalocauldal direction.

20. A method according to claim 19 comprising the steps of:
providing a device responsive to changes in curvature in a first direction;
placing the device on a subject's abdomen and/or chest wall with the device's said first direction being substantially aligned with the subject's cephalocaudal direction;
and monitoring changes in the device's output.

21. A method according to claim 20 including:
aligning a pre-determined portion of the device with a feature on the subject's abdomen.

22. A method according to claim 20 wherein the said feature is an anatomical feature.

23. A method according to claim 20 or claim 21 including providing a location marking or location markings on the device for alignment of the transducer device relative to a specified feature or specified features of the subject's abdomen and/or chest wall.

24. Devices substantially as hereinbefore described with reference to the figures.

25. A method substantially as hereinbefore described with reference to the figures.
